(19) United States
(12) Patent Application Publication
(10) Pub. No.: US 2024/0315779 A1
(43) Pub. Date: Sep. 26, 2024

(54) SYSTEMS AND APPARATUSES FOR FOR NAVIGATION AND PROCEDURAL GUIDANCE OF LASER LEAFLET RESECTION UNDER INTRACARDIAC ECHOCARDIOGRAPHY

(71) Applicant: KONINKLIJKE PHILIPS N.V., EINDHOVEN (NL)

(72) Inventors: VIPUL SHRIHARI PAI RAIKAR, SOMERVILLE, MA (US); KUNAL VAIDYA, BOSTON, MA (US); MICHAEL ANDERSON, COLORADO SPRINGS, CO (US); NATHAN C. FRANCIS, COLORADO SPRINGS, CO (US); BENOIT MORY, MEDFORD, MA (US); PASCAL YVES FRANÇOIS CATHIER, ASNIÈRES-SUR-SEINE (FR)

(21) Appl. No.: 18/574,775

(22) PCT Filed: Jun. 29, 2022
(86) PCT No.: PCT/EP2022/067959

§ 371 Date: Dec. 28, 2023

(30) Foreign Application Priority Data
Jun. 29, 2021 (EP) 21290044.3

(51) Int. Cl. A61B 34/20; A61B 8/00; A61B 8/08; A61B 8/12; A61B 18/00; A61B 18/24

(52) U.S. Cl. CPC A61B 34/20; A61B 8/085; A61B 8/0883; A61B 8/12; A61B 8/4245; A61B 8/463; A61B 18/24; A61B 2018/00369; A61B 2018/00577; A61B 2034/2063

(57) ABSTRACT

Systems and apparatuses for a resection procedure are provided. The apparatus includes an Intracardiac Echocardiography (ICE) probe for use in a Transcatheter Aortic Valve Replacement (TAVR) procedure, the ICE probe; and a processor device coupled to the ICE probe to provide positional feedback to a user about the ICE probe position as the ICE probe is positioned manually within cardiac anatomy wherein the processor device is configured to implement a model to provide guidance to manually position the ICE probe based on anatomical recognition of the cardiac anatomy wherein a manually positioned ICE probe is located at a position in the cardiac anatomy to enable capture of a view of a target cardiac anatomy in combination with the use of a catheter used while performing the TAVR procedure.

### TECHNICAL FIELD

The technical field generally relates to endovascular procedures and more particularly relates to systems and apparatuses for imaging, image-guided navigation, and recognition of cardiac anatomy while performing a resection using a laser energy device under intracardiac echocardiography.

### BACKGROUND

The use of Machine Learning (ML) applications is becoming more prevalent and recognized for applicability in various surgical phases; with advancements in ML application potential new applications are feasible such as the use in anatomical recognition in surgical procedures with different intraoperative data inputs such as video and instrument type to assist a medical provider in a surgical procedure.

Structural heart disease (SHD) is one of the fastest-growing fields in interventional radiology space which is a relatively new sub-specialty of interventional cardiology. The use of ML applications can play a part in this sub-specialty field to establish improvements in the standard of care for cardiac pathologies such as in the valvular disease domain.

Valve-in-valve repair has become deemed acceptable as a treatment for patients who have received a prior bio-prosthetic implant to prevent future valve failures and to mitigate the risk of complications such as coronary obstruction. From an imaging and device implantation perspective, the standard of care has migrated to performing these procedures purely on interventional x-ray fluoroscopy in a catheter lab environment. As these procedure numbers keep increasing and reinterventions become necessary, the impact of potential risks associated with reinterventions is an issue. During the procedure interventional x-ray alone is insufficient for monitoring of adverse events, such as pericardial effusion, which can lead to potentially fatal outcomes. Also, a vital necessary component of the imaging is the live visualization of the valve anatomy, particularly the leaflets. This imaging can currently be done only with some form of live ultrasound imaging. While typically, in structural heart procedures, live transoesophageal (TEE) ultrasound is used, there is an effort to move away from this as the need for general anaesthesia is eliminated. Transthoracic ultrasound (TTE) is also used but finding the right views can be challenging and highly dependent on patient anatomy as well as operator skill. Intracardiac echocardiography (ICE) has the potential to provide live ultrasound guidance and overcome the challenges of its traditional counterparts.

However, the use of current medical instruments that utilize cutting mechanisms presents certain drawbacks that when implemented intravascularly to successfully utilize ICE for this procedure. A newer methodology is required to enhance a safe user experience for both the patient and the non-traditional user of ultrasound imaging (i.e., the interventional radiologist). The cardiac cycle and rhythm introduce various types of motion which can affect the stability of the device as well as the images it produces. For a procedure that requires accurate and precise maneuvering of therapeutic devices, it is important to provide as much stability in the field of view as possible. In addition, for a non-traditional user of ultrasound, guidance, and feedback of relevance in both ultrasound as well as x-ray imaging can assist in reducing the cognitive load.

It is desirable to implement a system to stabilize the ICE probe, methods to provide navigation guidance and device trajectories with respect to key anatomical landmarks, and a method to successfully analyze the interaction of the resectioning device with its intended target, the valve leaflet.

The following disclosure provides these technological enhancements, in addition to addressing related issues.

### BRIEF SUMMARY

This summary is provided to describe select concepts in a simplified form that are further described in the Detailed Description. This summary is not intended to identify key or essential features of the claimed subject matter, nor is it intended to be used as an aid in determining the scope of the claimed subject matter.

The various embodiment describes apparatuses and systems to stabilize the ICE probe, to provide navigation guidance and device trajectories with respect to key anatomical landmarks, and to analyze the interaction of the resection device with an intended target, the valve leaflet.

An apparatus for use in an intracardiac procedure, the apparatus comprising: a processor device configured to provide positional feedback about a position of an intrabody ultrasound probe head as the intrabody ultrasound probe head is positioned within a cardiac anatomy to capture a view of a target anatomy of the cardiac anatomy. The processor device is configured to generate the positional feedback based on a reference position of the ultrasound probe head within the anatomy wherein the reference position comprises a position from which the intracardiac ultrasound probe captures a desired view of the target anatomy using an ultrasound field of view; and the apparatus comprising an output communicatively coupled to the processor device, the processor device further configured to provide at the output a feedback signal based on the positional feedback.

The apparatus can comprise a user interface configured to receive the feedback signal and provide an indication of the positional feedback to a user. The positional feedback may be further based on a current position of the intrabody ultrasound probe head within the cardiac anatomy and. The processor device can have an input for receiving the current position data and/or the reference position or any other data to be used by the processor device.

The reference position can comprise a position with which the intracardiac ultrasound probe captures the desired view of the target anatomy and an intrabody tool for use during the intracardiac procedure.

The processor device may be further configured to generate the positional feedback further based on structural characteristics of the target anatomy and one or more of: the field of view and the desired view. For example, a particular view showing the target anatomy from a particular perspective (e.g. a valve with all leaflets and annulus). A model may be implemented to recognize a predetermined view from current views and define the desired view based thereon.

The processor device may be further configured to: predicting a safe zone in the form of a virtual volume around the reference position, the safe zone encompassing positions the ultrasound probe head can reside in during the cardiac procedure while providing an acceptable one of the desired view; and optionally, determine whether a deviation of the current position from the safe zone occurs. The soft zone may be spherically shaped centered around the reference position.

The processor device may be configured to determine a similarity measure representative of a similarity between a current view of the intrabody target obtained with the intrabody ultrasound probe and a predetermined view of the target anatomy and determine the current view to be the desired view based on the similarity measure to therewith determine the reference position to be the current position pertaining to the corresponding current view. The processor device may be further configured to: determine the reference position based on a similarity measure exceeding a predetermined value, the similarity measure being representative of a similarity of a view of the target anatomy and, optionally the intrabody tool captured by the intrabody ultrasound probe at a position and a predetermined view of the target anatomy and, optionally the intrabody tool. The similarity measure can be a confidence score or other score indicating a degree of similarity. The predetermined view may be a view defined by protocol for a particular intracardiac procedure.

The processor device may be configured to determine the safe zone as defined herein based on the similarity measure as defined herein, wherein the volume of the safe zone increases with increasing similarity measure.

The volume of the soft zone is determined further based on a set of positional tolerances defined to accommodate an extent of motion of the intrabody ultrasound probe head without loss of the desired viewed of the target anatomy.

The processor device may be further configured to: receive an image of an imaging device; generate a representation of the safe zone for overlay with the image; and generate the positional feedback based on the image and the representation of the safe zone. The image can be an X-ray image such as 2D X-ray image. Hence an X-ray image overlayed with soft zone representation may be comprised within the positional feedback. The feedback may be displayed to a user.

The processor device may be further configured to: generate, during the intracardiac procedure, the positional feedback comprising one or more of: a representation of the safe zone; the deviation of the current position from the safe zone, instructions for repositioning the intrabody ICE probe head to within the safe zone and instructions to reacquire a reference position.

The processor device may be further configured to: apply a registration algorithm to determine the three-dimensional orientation of the intrabody ultrasound probe within the cardiac anatomy.

The disclosure provides for a computer implemented method for use during an intracardiac procedure the comprising the steps of: providing positional feedback about a position of an intrabody ultrasound probe head as the intrabody ultrasound probe head is positioned within a cardiac anatomy to capture a target anatomy of the cardiac anatomy; using a model, generating the positional feedback based on a reference position of the ultrasound probe head within the anatomy wherein the reference position comprises a position from which the intracardiac ultrasound probe captures an desired view comprising the target anatomy to be treated during the intracardiac procedure using an ultrasound field of view; and provide a feedback signal based on the positional feedback

The disclosure further provides for a computer program product comprising computer readable code which when run on a computer causes a processor device or apparatus as defined herein to perform the steps of any one of the methods described herein.

The current disclosure also provides (computer implemented) methods with steps corresponding to those the processor devices are configured to perform as define herein before.

An apparatus for stabilizing a probe head at the distal end of an Intracardiac Echocardiography (ICE) probe in a vessel lumen during an intracardiac procedure, the apparatus comprising an inflatable balloon device disposable about the probe head upon deployment within the vessel lumen; the inflatable balloon having a first state for insertion of the inflatable balloon into the vessel lumen together with the probe head during the intracardiac procedure and the inflatable balloon device having a second state to stabilize the probe head within the lumen, wherein the apparatus is configured such that the inflatable balloon can be inflated to cause it to change from the first state to the second state while deployed in the vessel lumen together with the probe head to therewith cause the inflatable balloon to exert a compression force to part of the probe head and at least part of the vessel lumen wall surrounding the probe head to thereby provide the stabilization. The apparatus may have a lumen extending along an elongated shaft to which the balloon is attached where the lumen is hollow and provided to inflate and deflate the balloon.

The inflatable balloon may comprise a first opening at its distal end and a second opening at its proximal end and a lumen connecting the first and the second opening, the lumen being configured to accommodate at least a part of the probe head. This way the blood flow is not obstructed substantially and stabilization may be increased due to less blood pressure disturbance.

The diameter of the inflatable balloon may be sized to about a diameter of the vessel lumen, such as for example that of the Vena cava lumen when in inflated state.

The inflatable balloon of the balloon device may comprise a compliant material. This may prevent or reduce overstretching of the vessel lumen when the inflatable balloon is in the second state.

The inflatable balloon device may be an occlusion balloon type of device. Simple known balloons can thus be employed.

The inflatable balloon may be designed to be shaped in the second state such that when deployed in the vessel lumen in the second state a channel for blood flow extends from a proximal end to a distal end of the balloon.

The apparatus may further comprise the ICE probe

The inflatable balloon device, when in the second state, may be configured to reduce or prevent movement of the probe head in the vessel lumen by the compression force of the ICE probe head against the vessel lumen.

The apparatus may be configured to stabilize the probe head such that a reduction or prevention of motion of the probe head is obtained where the motion is caused by one or more actions chosen from the group consisting of: blood flow or blood pressure in the vessel lumen, cardiac motion, and user interaction.

The apparatus, may be configured such that in response to the balloon device stabilizing the probe head in the vessel lumen, a laser catheter is enabled to perform ablation for the intracardiac procedure by a single user.

In yet another exemplary embodiment, a system to assist in a valve resection procedure is provided. The system includes a Neural Network (NN) model to detect and to predict three-dimensional landmarks in a valve resection procedure for proper localization wherein the NN model is a semi-supervised trained NN based on a set of ultrasound anatomical images generated in a prior endoscopic procedure, and a processor device configured to implement the NN model by processing a set of images of cross-sectional views of leaflets in the valve resection procedure to monitor a grasping operation of a leaflet by a grasping mechanism and to provide confirmation of proper leaflet insertion during the grasping operation based on image comparisons of images of leaflet insertions during the grasping operation with cross-sectional views of leaflets contained in the NN model.

In at least one exemplary embodiment, a system that is provided includes the system with the processor device is configured to implement the NN model to track the motion of leaflets during the grasping operation for comparisons of aspects of leaflet motion to determine the proper leaflet insertion.

In at least one exemplary embodiment, a system that is provided includes the ML system with the processor device is configured to implement the NN model to compare pre-grasping leaflet motion versus post-grasp motion to determine the proper leaflet insertion.

In at least one exemplary embodiment, a system that is provided includes the ML system with the processor device is configured to implement the NN model to estimate pre-grasping motion versus post-grasping motion to determine the proper leaflet insertion.

In yet another exemplary embodiment a computer implemented method to assist in a valve resection procedure is provided. The method makes use of the neural networks as defined herein before in paragraphs 0027 to 0030.

Corresponding to any of the computer implemented methods defined herein the current disclosure defines computer program products comprising computer readable code which when run on a computer implement the methods defined herein. The code may be stored on non-transitory computer readable medium

Furthermore, other desirable features and characteristics of the system and method will become apparent from the subsequent detailed description and the appended claims, taken in conjunction with the accompanying drawings and the preceding background.

### BRIEF DESCRIPTION OF THE DRAWINGS

Examples for implementing the present invention will hereinafter be described in conjunction with the following schematic drawings, wherein like numerals denote like elements.

FIG. 1 illustrates a diagram of an exemplary process of determining a pose of an ICE probe in a coordinate system, such as an imaging coordinate system;

FIG. 2 illustrates a diagram for an exemplary process for positioning of an ICE probe in an imaging coordinate system such that the ICE probe provides a desired field of view and a process for checking and/or correcting the position of the ICE probe;

FIG. 3. illustrates a diagram of an exemplary inflatable balloon device to secure a chosen position of an ICE probe having a desired field of view;

In FIG. 4, illustrates a diagram of an ICE probe secured at a position within the heart using an inflatable balloon;

FIG. 5 illustrates an elasticity of an inflatable balloon used to secure an ICE probe position;

FIG. 6, illustrates a diagram of an exemplifying safe-zone in the right atrium (RA) in which safe zone the ICE probe head can reside to provide a desired view in combination with the laser device in accordance with an embodiment;

FIG. 7 illustrates a diagram of a desired field of view showing a heart valve that triggers a fail-safe process to disable the laser device and provide sensory feedback to the operator in accordance with an embodiment;

FIG. 8 illustrates an exemplary diagram of a common coordinate frame with a reference for navigating an ICE volume with anatomical landmarks in accordance with an embodiment;

FIG. 9 illustrates an exemplary diagram of a combination of a neural network (NN) trained system in multi-task learning for predicting anatomical landmarks in accordance with an embodiment;

FIG. 10 illustrates an exemplary diagram of a Spatial transformer network (STN) to process inputs from the encoder of a localization network in accordance with an embodiment;

FIG. 11 illustrates an exemplary diagram of a machine learning (ML) system for enabling a type of multitask learning network for detecting devices in three-dimensional (3D) volumes in accordance with an exemplary embodiment;

FIG. 12 illustrates an exemplary diagram of a piecewise approximation for combining landmarks from x-ray to ultrasound to improve the distal shape and catheter segmentation of catheter-like devices in accordance with an embodiment;

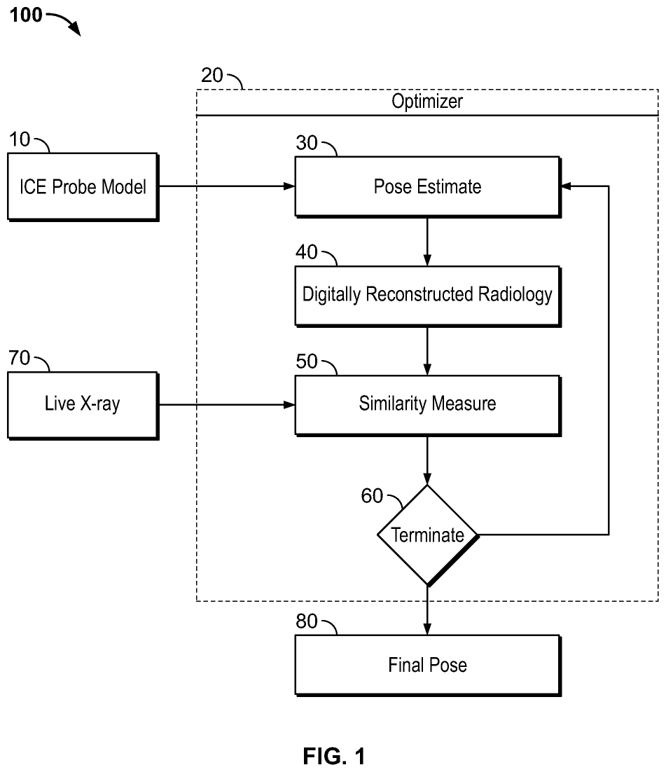

FIGS. 1A, 13B, and 13C illustrate exemplary diagrams of leaflet motion estimations for a leaflet resection procedure in accordance with an embodiment; and

FIG. 14 illustrates a laser ablation system, ICE probe feedback system, and imaging system in accordance with an embodiment.

### DETAILED DESCRIPTION

The following detailed description is merely illustrative in nature and is not intended to limit the embodiments of the subject matter or the application and uses of such embodiments. As used herein, the word “exemplary” means “serving as an example, instance, or illustration.” Thus, any embodiment described herein as “exemplary” is not necessarily to be construed as preferred or advantageous over other embodiments.

The embodiments described herein are exemplary embodiments provided to enable persons skilled in the art to make or use the concepts defined herein and not to limit the scope of the claims. Furthermore, there is no intention to be bound by any expressed or implied theory presented in the preceding technical field, background, summary, or the following detailed description.

The concepts of the invention will now be explained in more detail using the example of TAVR intravascular procedure guided by an ICE probe and a 2D X-ray system using the figures as provide herein.

However, while embodiments are described for guiding and performing TAVR procedures using an ICE probe, the concepts may also be used with other intrabody imaging devices such as for example TEE probe. Furthermore, the concepts described may also be used in other intrabody interventional procedures, such as intravascular and intracardiac procedures, in which intrabody US imaging is used. Thus, the concepts may be used for intracardiac structural heart repair procedures. For example, the methodology described is at the very least applicable to procedures directed to all valve repair procedures including the other three valve leaflets in the anatomical region in equal measure and may also apply to a host of other intravascular or intracardiac procedures. However, yet other intracardiac procedures may benefit from the concepts described herein. It is noted however, that due to the complicated construction of valves and the rather vigorous motion induced by the pumping of the heart, valve related procedures are generally quite complicated to perform, require a strict protocol and benefit considerably from the concepts described. Also, while the concepts are described for application of an ICE probe in the context of laser resectioning of aortic valve leaflets, they are not limited to laser resectioning, or resectioning in general of either aortic valve leaflets or other anatomical regions.

The TAVR procedure allows for the implanting (i.e., replacing) of a heart valve without having to open the chest cavity. The resultant minimal invasive surgery for a heart valve replacement makes surgical valve replacement a more feasible treatment plan. This is because TAVR can now be considered an option for patients considered at intermediate or high risk of complications from traditional open-chest surgical aortic valve replacement.

However, there are differences in both approaches; for example, in open-chest replacement surgery, the degraded valve is completely removed. In TAVR, the damaged native valve is left in place. The valve can have anatomical abnormalities, calcification, or infection. However, inserting a new valve over the native valve can cause complications in the TAVR procedure, including valve migration, valve embolization, paravalvular leakage, and blockage of the coronary arteries restricting blood flow to the heart.

The TAVR procedure allows for the implanting (i.e., replacing) of a heart valve without having to open the chest cavity. The resultant minimal invasive surgery for a heart valve replacement makes surgical valve replacement are more feasible treatment plan. This is because TAVR can now be considered an option for patients considered at intermediate or high risk of complications from traditional open-chest surgical aortic valve replacement.

Therefore, deploying a TAVR valve on top of the existing damaged valve may not be performed because of the procedure's expected complications. To alleviate the complications from implantation of a new valve, a catheter may be used to remove old valve leaflets at the location to prepare the implant site for a cleaner valve deployment and operation

For guidance of the TAVR procedure (intravascular) an ICE probe is placed directly inside the heart or a part thereof a patient. During the procedure, the position of the ICE probe inside the heart is crucial for a clear view of the target anatomy (in this case the valve to be resected), and any tools used in the procedure by the ICE probe and the imaging system. A good or optimal desired position of the ICE probe for this purpose requires awareness of the probe location and orientation (the probe pose) in the heart anatomy along with the live view of the target anatomy provided by it.

This present disclosure describes systems, apparatuses and methods that enable positioning and/or stabilization of an ICE probe used in an intravascular procedure and particularly an intracardiac procedure such as for example a TAVR procedure, with navigation guidance and device trajectories with respect to anatomical landmarks, that facilitate the interaction of the re-sectioning device with its intended target (i.e., the valve leaflet).

In the various exemplary embodiments, the present disclosure describes systems and apparatuses that provide enhance situational awareness of the current spatial location of the ICE probe and for optimum anatomical viewing to improve visualization by the medical provider when performing an endoscopic procedure; an intravascular procedure or, in particular, an intracardiac procedure.

In the various exemplary embodiments, the present disclosure describes systems and apparatuses that predict a safe zone within the cardiac anatomy for the ICE probe head. The safe zone may be used to guide the user in placement or replacement of the ICE probe head to provide a desired view of the target anatomy and any tools if applicable. Also, the safe zone may help confining the location of the ICE probe head within a defined zone to ensure desired imaging during execution of the procedure performed. Furthermore, if a deviation of the probe from his zone occurs, the deviation can be detected, and the operator may be notified via sensory feedback.

In some of the various exemplary embodiments, the present disclosure describes systems and apparatuses that provide better stability of the ICE probe head (which includes the US imaging array) to reduce or minimize the effects of e.g., cardiac motion that may interfere in performing imaging in any of the procedures. Also, once the probe head is stabilized in the desired position, a single provider can determine the three-dimensional (3D) ultrasound (US) volume and manipulate the tip and trajectory of the laser catheter in the 3D US volume.

The described application of the ICE probe enables an endoscopic procedure to be performed by a single medical provider since the ICE probe once positioned and stabilized can free up the single medical provider to perform the other endoscopic tasks such as the ablation by the laser catheter that ordinary would require an additional set of hands to perform.

The concepts of the invention will now be explained in more detail using the example of a TAVR procedure guided by visualization using an exemplifying guidance system as defined herein making use of an ICE probe and a 2D X-ray system (fluoroscopy).

Thus, in an exemplifying TAVR procedure in which the aortic valve is the target anatomy, an ICE probe is inserted in a heart of a patient situated on a table of a catheter lab. The ICE probe may be catheter type device having a shaft section, an ICE probe head at a distal end of a shaft section, and ICE probe controller at the proximal end of the shaft section. The ICE probe head comprises the US imaging elements and may be operable to provide a 2D and/or 3D US imaging. The ICE probe is connectable to a controller which can collect, store, and process US image data. The ICE probe controller is further configured to control a pose (position and/or orientation) of the ICE probe and/or, optionally, imaging parameters to adjust its field of view, type of imaging (intensity, doppler) etc. For example, the controller can include a user controllable handle or an interface operable by a robotic system or the like disposed at the proximal end of the catheter. The controller can be used to reorient and/or relocate the ICE probe in the heart. Such catheters are known perse and will not be further described for brevity.

The ICE probe is thus advanced through the vasculature of the patient into the heart. For example, the ICE probe may be entered via the femoral vein to be positioned in the vena cava or part thereof such as the superior vena cava or inferior vena cave. A 2D X-ray system (for example a C-arm based 2D X-ray system), which is a commonly used imaging system within the catheter lab to guide operative procedures, is positioned and oriented such that its field of view can record intraoperative 2D images of the heart where the images include the target anatomy, parts of the ICE probe (preferably the ICE probe head) and, possibly, also parts of other intrabody tools used during the TAVR procedure.

The guidance system is configured such that the ICE probe pose can be registered to the X-ray systems coordinate system. Therewith the ICE probe pose in the 2D X-ray image is known or can be determined continuously or periodically or on demand. The X-ray system coordinate system may be any coordinate system as known in the art. Such registration ensures that during a procedure any change of pose of the ICE probe with respect to the 2D X-ray system may be known to the user (physician or other system such as robotic system).

FIG. 1 illustrates a diagram of an exemplary registration method and system. The optimizer module 20 is configured to generate a final pose 80 estimation of an ICE probe within the X-ray system coordinate system. In a first step, the pose estimation module 30 receives (for example from a memory) a 3D ICE probe model which corresponds to the ICE probe used in the procedure and, using the model, generates an output of pose results for the ICE probe for example, within (or with respect to) the X-ray system coordinate system. The 3D ICE probe model may be generated by its imaging using a 3D imaging modality such as for example CT or MRI or the like. Alternatively, another type of 3D modelling technique may be used to obtain the model. The output of pose results is sent to and received by a reconstruct module 40 which, based on the output of pose results, generates a radiograph or digital reconstruction representing the 3D ICE probe within a 2D X-ray image. The digital reconstruction may thus represent the ICE probe as viewed along an estimated direction with respect to the X-ray system coordinate system. The module 40 sends the digital reconstruction to the similarity module 50. The similarity module 50 receives the digital reconstruction and receives a 2D image from the X-ray system where the X-ray image shows the real ICE probe while inserted in an anatomy of the patient and along an X-ray system imaging direction. The similarity module 50 then compares the image to the digital reconstruction to determine a similarity measure between the two which measure is sent to the decision module 60. The decision module 60 determines if the similarity measure is of a sufficient amount to conclude that the digital reconstruction and image are close enough. If the similarity measure is deemed not of a sufficient amount, the decision module 60 determines not to terminate the process flow but to return to the pose estimation 30 by sending a feedback type signal to the pose estimation module 30 which upon receipt of that feedback signal refines the pose estimation and outputs a further pose estimation output to the module 40 to try for pose results that result in an increased similarity measure. The steps performed in modules 30, 40, 50 and 60 are repeated until the module 60 determines the similarity amount to be sufficient to terminate the process flow. At such instance the latest estimated pose corresponding to the sufficient similarity is output as the final pose 80. The optimizer module thus provides a current pose of the ICE probe in the X-ray system coordinate system. A more detailed description of the registration method is provided in publications in Medical Image Analysis 16 (2012) 38-49 and WO2011/070477 which are incorporated by reference.

The optimizer of FIG. 1 provides convenient registration of ICE probe pose with the X-ray X-ray system without any pose trackers. Other registration modules may employ other methods including for example tracker systems capable of tracking sensors based on electromagnetic principles or US principles etc. In such case the X-ray space and US space are registered to each other by using fiducials that are visible in the X-ray imaging space while they have known location in the X-ray space via the tracking by the tracker system. Tracking the sensors on a tool such as ICE probe in X-ray space may then be transformed in a pose of the tool in X-ray image.

The optimizer module of FIG. 1, or in a more general sense, any registration module, may be triggered upon request by the user directly, or indirectly. For example, when the user instructs the system to record an X-ray image, the system may also trigger the optimiser to run using the newly recorded X-ray image. Alternatively, or additionally, by repeatedly or continuously performing the optimizer module a continuous registration of ICE probe position (location and orientation) is obtained without any user interference.

The registration enables knowing the pose of the ICE probe within the X-ray system coordinate system. Additionally, since the ICE probe may be or is calibrated such that its field of view properties are known with respect to its US imaging sensors in the ICE probe head and the positioning of the X-ray system (i.e., orientation of the C-arm within the imaging coordinate system) during measurement of any 2D X-ray images is also known, the US image may be or is also registered with the X-ray image.

In one embodiment the user now operates the 2D X-ray imaging system such that it has a field of view capturing at least the ICE probe and heart to help guide the insertion of the ICE probe into the heart. While the ICE probe will have a good contrast in an X-ray image, soft tissue of the heart is usually less contrasted and to help increase contrast fluoroscopy can be employed.

Next, a position and orientation (pose) of the ICE probe within the hart is searched that provides a desired or optimal view (US image) of the target anatomy using the ICE probe. The “desired or optimal position” will be referred to as the “reference position”, the corresponding desired or optimal orientation will be referred to as the “reference orientation” and the corresponding desired or optimal pose will be referred to as the “reference pose”. This search may be done by manipulation of the ICE probe over multiple poses until the desired or optimal view is captured by the ICE probe. Several methods can be used to find the reference position and orientation as will be described herein below.

A desired or optimal view for the TAVR procedure may be predetermined from protocol. For example, such view may be defined to capture all leaflets of the valve and the annulus. Once the desired or optimal view has been found by ICE probe head manipulation and comparison of desired and current view, the corresponding position, and/or orientation determined using a registration method as described herein before. For example, an X-ray may be recorded at that point in time to have the optimizer module of FIG. 1 use it to determine the pose of the ICE probe head. After all, for example the final pose 80 provides such position of the ICE probe. Thus, the ICE probe optimal position and/or orientation with respect to an (optionally already optimised) X-ray imaging system may be determined. The so determined position and orientation are now stored as the reference position and/or the reference orientation

At this point of the procedure a reference position and/or orientation of the ICE probe head is known in the imaging coordinate system (i.e. any X-ray image) from which the ICE probe can have a desired or optimal field of view for the TAVR procedure.

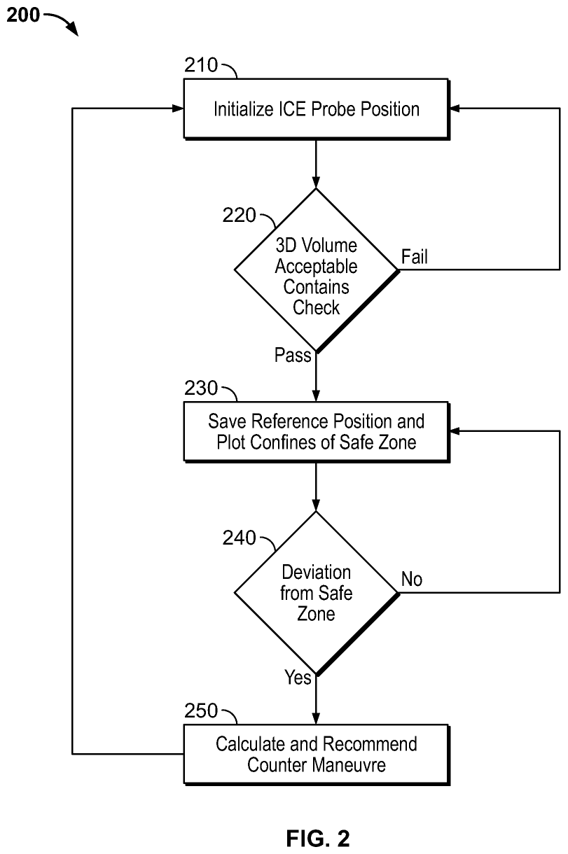

FIG. 2 illustrates a diagram of an exemplary more detailed process for finding the reference position and/or orientation. The process can be performed by the ICE probe guidance and placement system in accordance with an embodiment. In FIG. 2, at the step 210 an initial pose (location and orientation) of the ICE probe head in the X-ray system coordinate system is recorded. This initial pose may be a current position and orientation of the ICE probe head as determined with a registration method described herein above. In a next step 220 of the method it is determined whether the initial position qualifies as a reference position, or whether the ICE probe head requires repositioning (e.g., physical movement in the form of translation and/or (re)orientation) to obtain become a reference position. The determination is based on a check of acceptance criteria relating to the comparison of e.g., the ICE probe heads' current field of view and resulting US image to the predetermined desired or optimal field of view and US image chosen for use during the TAVR procedure. The desired and optimal field of view is chosen to be suitable for US imaging of the target anatomy and, optionally, any interventional intrabody tools for use during the TAVR procedure such as leaflet grasping tools and or resectioning tool. Note that the ICE probe head is generally better capable of providing the desired or optimal view of the target anatomy as that is soft tissue. The tools used during the TAVR procedure are generally better visible in the X-ray imaging system field of view and image. As also mentioned herein before, the desired or optimal placement of the ICE probe head with respect to target anatomy and tools used during the procedure requires awareness of the ICE probe head's current position in the X-ray system coordinate system with the available live views of the anatomy that can be captured by the ICE probe and, optionally, the imaging system.

One way of finding the reference position is described in the patent application U.S. Pat. No. 16,979,542, published as Us20210000446, entitled ULTRASOUND IMAGING PLANE ALIGNMENT GUIDANCE FOR NEURAL NETWORKS AND ASSOCIATED DEVICES, SYSTEMS, AND METHODS which is incorporated by reference. This application describes a guidance system and method for obtaining a medical image where the system includes a processor configured to obtain a motion control configuration for repositioning an imaging device from a first imaging position to a second imaging position with respect to a subject's body, the motion control configuration obtained based on a predictive network, an image of the subject's body captured while the imaging device is positioned at the first imaging position, and a target image view including a clinical property; and a display in communication with the processor and configured to display an instruction, based on the motion control configuration, for operating a control component in communication with the imaging device such that the imaging device is repositioned to the second imaging position. The imaging device may be an ICE probe and the motion control configuration comprises instructions for a robotic system or a physician to relocate and/or reorient the ICE probe head and therewith obtain an improved field of view with the ICE probe.

The method and system described are based on neural network models trained to be capable of checking, using acceptance criteria, and deciding whether a current ICE probe head field of view or image is similar enough to a predetermined desired field of view or image to be used for a procedure. If not, the acceptance criteria are determined to have been failed during check at step 220, and instructions may be given to a user (physician or robotic system) to relocate and/or reorient (i.e., change the pose of) the ICE probe head. In addition, instructions are given to reperform step 210 to (re)initialize the ICE probe head position and/or orientation and perform a further check for the ICE probe image with new pose. In this way an iterative process guides the user in reposing the ICE probe head towards one that causes the system in step 220 to decide that the acceptance criteria have been fulfilled. At that instance, the corresponding initialized ICE probe head position and/or orientation is determined (for example using the optimizer module procedure or other registration method) and stored in step 230 for further use. Thus, after exit of step 230 an ICE probe pose in the X-ray system coordinate system may be obtained and stored as a reference pose from which a desired or optimized ICE probe view is enabled.

In an alternative, or slightly adjusted and less preferred method the user may manually manipulate the ICE probe without the acceptance criterion check as described herein before, while viewing the ICE probe image until the user decides the optimal ICE probe image is obtained. At such instance he may indicate the system, via user interface, to record the corresponding position of the ICE probe in the imaging system coordinate system as the reference position in step 230. The recording can be asked for by the user by running a registration method herein.

The reference position and/or orientation so determined may be used during the remainder of the TAVR procedure for multiple purposes. At step 230, the reference position and/or orientation are saved as well as the deviation limits.

The defined limits may be used to define (calculate) a “soft zone” in the X-ray system coordinate system around the refence position in which soft zone the ICE probe head may be located and still be able to provide an acceptable desired view of the target anatomy. For example, at step 230, a virtual three-dimensional (3D) volume (also referred to as a “safe zone”) is generated (e.g., computed) surrounding the ICE probe tip in which the ICE probe can reposition and/or reside during a procedure without the system generating the above described warning signal. This safe zone effectively is bordered by the defined limits of repositioning as mentioned in the previous paragraph.

The safe zone is based on the reference position, the ICE probe field of view and the target anatomy and its location in the field of view. The volume of the zone may be predetermined based on the procedure and must accommodate an acceptable motion of the probe head as caused by the heart, target anatomy etc. during the TAVR procedure. For example, a wide field of view may tolerate more motion before the target anatomy is lost from the field of view than a narrower field of view. Likewise at equal field of view width, a target anatomy at greater depth within that field of view may tolerate more motion. Thus, for example the volume of the soft zone may be dependent on one or more of: width or volume of field of view and depth of target anatomy within field of view. Furthermore, since the field of view may be chosen dependent on the type of target anatomy to be observed as required by a procedure to be performed, the soft zone may also depend on the type of procedure and/or target anatomy. In one embodiment, the volume of the soft zone is modelled as a sphere-like shape with a diameter dependent on the above parameters.

Alternatively, or additionally, the deviation limits and soft zone the deviation limits and/or soft zone may be based on (computed using) a similarity measure representative of a similarity of live 3D ultrasound image recorded at the reference position and/or pose) and the ideal image that captures the anatomy of interest (i.e. the leaflet valve). For example, such measure may result from the method used to determine the reference position as described herein before with respect to FIG. 2. The measure may for example comprise or based on a confidence score with higher score indicating increased similarity. However, others can be used. How such measures for comparison of measured and desired images can be obtained is known in the art and will not be detailed herein.

The deviation limits, soft zone, e.g. volume and/or the diameter of the spherically shape soft zone may be based on the similarity measure or score. Thus, the higher the similarity measure, the larger the deviation limits and the larger the soft zone (e.g. diameter) may be.

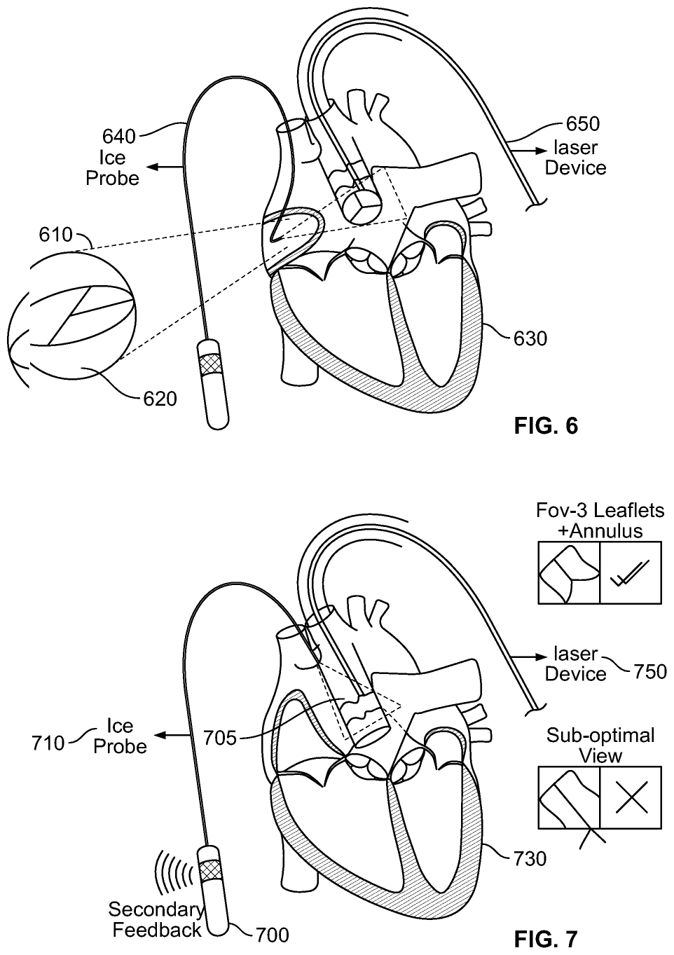

An indication of the soft zone may be output to the user. Preferably an icon like indication is overlayed on the X-ray image. The icon may be a circle or sphere on a 2D image or even on a 3D image. The icon preferably also indicates the reference position as the centre and, optionally also an indicator representing the pose of the ICE probe head, or even a direction of the field of view of the ICE probe. The latter may be obtained from the calibration of ICE probe field of view with respect to its imaging sensors. For example, the indicator may be a line extending along the direction from centre to circle or sphere. See also description of FIG. 6 herein below for an example. The icon may thus be displayed on a display for the user together with the X-ray image. The configured unique soft zone is therefore centered on the patient-specific anatomy as for example imaged with X-ray imaging in this case.

Knowing the safe zone with respect to the X-ray system coordinate system is advantageous. For example, at a particular time during a procedure, the ICE probe position and/or orientation may be lost due to movement. A visually assisted repositioning and/or reorientation of the ICE probe head may be guided with the safe zone, either with or without additional reposing parameters calculated and provided by the system to the user as defined herein above. Furthermore, a new X-ray imaging orientation may be chosen and the soft zone easily adjusted (by calculation) for that new X-ray image since the soft zone is determined in the X-ray system coordinate system. Thus, after new X-ray imaging, the ICE probe pose may be checked and adjusted if necessary, with simplified new desired view finding for the ICE probe as the head may be conveniently positioned in the soft zone.

In one embodiment, once a reference position and/or orientation has been determined in step 230, the actual pose of the ICE probe during the remainder of the procedure is compared to and tracked by the system with respect to the reference pose. As long as the actual pose so tracked does not deviate from the reference pose beyond defined deviation limits because of any ICE probe head repositioning (relocation, reorientation or both) due to patient and/or procedurally induced motion, the system allows the user to continue the TAVR procedure without warning or blocking measure. If, however, the ICE probe becomes repositioned beyond the defined limits, the system generates a deviation signal that may be output to the user and/or trigger calculation of reposing parameters with instructions of how to reposition and/or reorient the ICE probe head to bring it within the limits of the reference position. Alternatively, the deviation signal may start the procedure of FIG. 2 anew again to define an entirely new reference position and/or reference orientation.

Thus, at step 240, the deviations are determined from the safe zone. The saved references and plot confines are updated by a feedback loop of input or the deviation information generated at step 240. Once completed, an algorithmic solution is used to calculate and recommend counter maneuvers for the ICE probe placement at step 250.

In some embodiments, the guidance system includes an anchoring system for example as described herein below. The guidance system and method described herein before may be used to provide an anchoring control system to the anchoring system. Such control signal may include an anchoring release signal when repositioning and/or reorientation of the ICE probe head is required. Alternatively, or additionally such system may include an anchoring engage signal when a reference position and/or orientation has been obtained and/or is to be maintained. The signals may be generated using the view finding elements described in FIGS. 1 and 2. Alternatively, a manual control may be implemented for dynamic anchoring of the ICE probe head. Differently generated control signals are envisaged. For example, based on imaging features in X-ray and ultrasound, any deviation of the probe head or loss of view can be used to send signals to the anchoring system and either prompt user to reposition or automatically reposition the probe.

In some embodiments, the guidance system can comprise an optional anchoring device for stabilizing the pose of the ICE probe head within an anatomy, for example around the reference position or within the safe zone. Such anchoring device may take the form of a mechanical system using a mechanism for clamping the ICE probe head within a vessel lumen. Exemplifying anchoring systems include deployable stents or inflatable balloons. An exemplifying anchoring system in the form of a balloon catheter system is described herein. Its purpose is to stabilise the ICE probe head around the reference position, but the use of the balloon catheter system is by no means limited to such stabilising such position. In principle any chosen position of the ICE probe may be stabilised and this may be done while guiding any intravascular or intracardiac procedure. With stabilising is at least meant reduce the chance of, or reduce the actual, translation or reorientation of the ICE probe head due to motional causes such as cardiac motion, blood flow and or user instigated movements compared to a situation without using the anchoring system. The anchoring device may be operable manually or may be operable automatically based on control signals from the guidance system as described herein before.

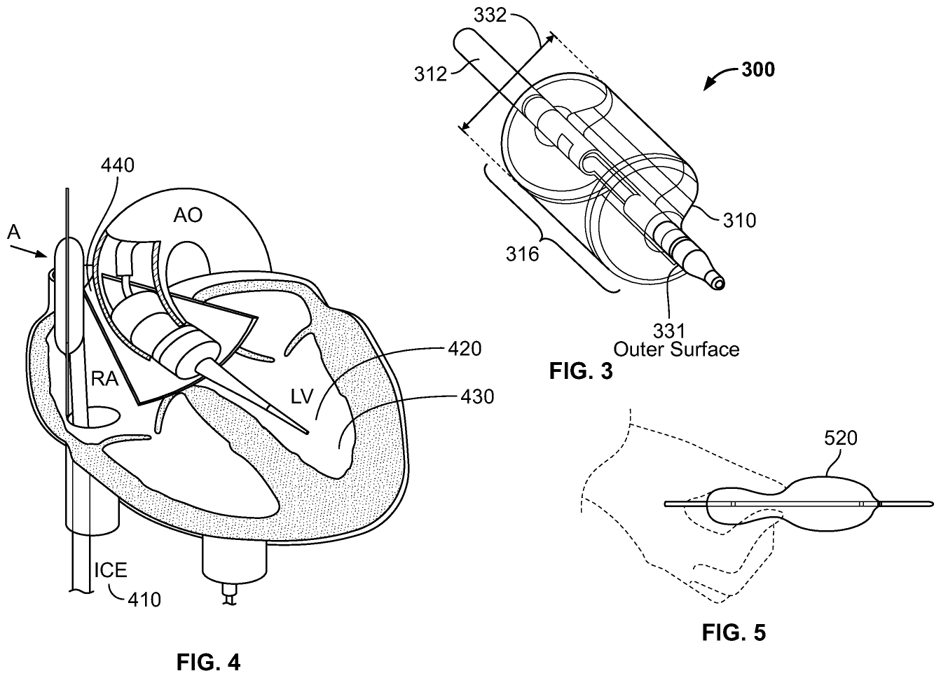

As an exemplary guidance system including an ICE probe and an anchoring device, FIG. 3 shows a diagram of a balloon catheter system implemented to position and secure the elongated shaft section 312 of an ICE probe 316, once guided to a desired position such as for example the reference position, at a position within an anatomy. The balloon catheter system includes an inflatable balloon 310 as part of the anchoring device configured to form an envelope around (in the vicinity of) the ICE probe head 316 upon inflation to therewith secure the ICE probe head in a position and/or orientation for the desired or optimum viewing of the target anatomy such as the valve leaflet.

When designed to be used for anchoring in a lumen for blood (or other fluid) flow, the balloon catheter system is preferably configured to not impede the blood flow during expansion of the balloon by enabling blood flow through a secondary lumen connecting the lumen spaces at the proximal and distal ends of the inflated balloon. The secondary lumen can be any opening for sustaining blood flow from proximal to distal end of the balloon. Thus, the secondary lumen can be integrated in the ICE probe catheter. Alternatively, or additionally, the secondary lumen can be part of or integrated in the balloon device or balloon when the balloon is expanded. Having the secondary lumen as part of the balloon device, is beneficial as it allows use of a regular ICE probe not having a secondary lumen for the interventional procedure. As said, this secondary lumen is beneficial when the ICE probe head is to be positioned or anchored within lumen within which blood flows during the procedure performed as with an artery or vein such as vena cava or aorta during a cardiac procedure such as for example the TAVR procedure.

In an exemplary procedural method, the ICE probe head 316 is stabilized in a manner to secure the position of the ICE probe head 316 in the lumen and at a position that minimizes tremors that can be realized by the ICE probe head and are caused by internal body cavity vibrations (or movements) from a beating heart. Upon a determination or discovery of the optimum, correct, or best available FOV in the lumen (e.g., the superior vena cava (SVC)) using for example the methods and systems as described herein before, the stabilizing process for the ICE probe would include inflation of the inflatable balloon. Preferably, when the balloon is to be used within a specific lumen such as vena cava, the inflatable balloon 310 in its inflated state is designed to have a diameter sized to about or slightly larger than the diameter 332 of the lumen such as the SVC. This may prevent overstretching of the lumen.

In an exemplary embodiment, the balloon device 300 is configured to be placed at least partly and preferably completely around the elongated shaft section 312 of the ICE probe head 316 that has an outer surface 331. For example the balloon device may comprise a probe lumen configured to accommodate at least part of the elongated shaft section 312 therein. The inflatable balloon 310 is located and disposed of about a portion of the outer surface 331 at the distal end of the elongated shaft section 312. The ICE probe imaging sensors may be within the envelope of the balloon in inflated state or outside of the envelope.

As described, the inflatable balloon 310 is placed in a first state (deflated or partially deflated) for insertion into the vessel lumen to travel down the vessel lumen and placed in a second state (inflated or partially inflated) to stabilize the ICE probe. The inflatable balloon 310 when inflated in the second state can exert a compression force to the distal end of the ICE probe that may be close to or include the ICE probe head 316. The compression force is caused by inflation of the inflatable balloon sized to the vessel lumen and pressing against the ICE probe head 316. The inflatable balloon 310 is sized to about a diameter of the vessel lumen to stabilize the head of the ICE probe head 316 in the second state. The inflatable balloon 310 is made of a compliant material that prevents overstretching of the vessel lumen in the second state. The balloon device 300 is configured using an occlusion balloon type of device. An example of a legacy product of an occlusion balloon is the PHILIPS® BRIDGE™ Occlusion balloon.

It is noted that after anchoring of the ICE probe head using an anchoring mechanism as described herein above, the ICE probe field of view as chosen using methods and systems as described herein before, may require adjustment because of some interference caused by the expansion and/or expanded state of the balloon. This adjustment may for example be done by controlling the 3D field of view (3D ICE) or 2D field of view (2D ICE) using the regular US imager control mechanisms.

With the ICE probe head 316 stabilized, a single user can perform the procedure in a yet improved way because no assistance is required to manually hold the ICE probe 316. Thus, for example the TAVR procedure when performed using a laser ablation resectioning device may be performed more easily or with improved effect by the single user as he is allowed to concentrate on the manipulation of the laser resectioning device to perform the ablation.

FIG. 4, shows a diagram of an exemplary combination of the balloon device as described herein and the ICE probe head with the ICE probe position secured once the probe has been guided to the optimum viewing position for example using the ICE probe head guidance system as described herein. In FIG. 4, the ICE probe head and balloon configuration 410 is shown to have entered the heart through the inferior vena cava traverse the right atrium (RA) and advanced to have the ICE probe head and balloon 460 located just in the superior vena cava. The actual ICE probe head is not shown as it is in this case located next to and behind the balloon. The ICE probe head is positioned such that its field of view 440 in the direction indicated with the letter “A” and represented by the truncated triangular plane includes the location of the aortic valve and in this case part of the catheter device snaked through the aorta (AO) and with its tip 420 entering the left ventricle (LV) 430. The field of view enables the visual imaging of the leaflet. The compression clamping of the ICE probe head in the SVC reduces the tendency of or even prevents the ICE probe from repositioning (displacement and/or orientation) due to motion that can happen from the blood flow, the medical provider interaction, or cardiac motion (i.e., the vibrations caused by heartbeats).

In FIG. 5, is a diagram that displays the elasticity of the balloon used to secure the ICE probe head of the ICE probe guidance and placement system in accordance with an embodiment. The balloon 510 is configured using a compliant, stretchable, and mouldable material, that when expanded is limited in size to prevent an overstretching of the SVC lumen of the puncture from a reaction force caused by compressing the ICE distal tip into the lumen or heart walls. An example of an inflatable balloon is the PHILIPS® Bridge Occlusion balloon. In an exemplary embodiment, the balloon device 300 can be configured in 80 mm in length and 20 mm in diameter with a maximum inflation volume of 60 cc. The U.S. Pat. No. 10,499,892 entitled TEMPORARY OCCLUSION BALLOON DEVICES AND METHODS FOR PREVENTING BLOOD FLOW THROUGH A VASCULAR PERFORATION, filed Mar. 16, 2016, and issued on Dec. 10, 2019 assigned to SPECTRANETICS® Corporation with inventors Ryan Michael Sotak, Grant Foy, Phil Aranas, Jay Harper is incorporated by reference.

In some embodiments the anchoring system is not used. In any case, whether used or not, FIG. 6, illustrates a diagram indicating a soft zone and a reference position in the right atrium (RA) of the ICE probe 640 in combination with a laser device as for example used with a TAVR procedure. The laser device 650 is positioned in the aorta and advanced towards the aortic valve. The safe zone is in the right atrium 630. It is represented in enlarged view by the spherically-shaped indicator with a diameter 610, a centre location 615 and a view direction indicator 620 for indicating the direction of the field of view of the ICE probe. The FIG. 6 shows the soft zone indicator in 3D perspective. In some embodiments of the method the soft zone is indicated in the X-ray image. In such case the soft zone may be represented by a 3D indicator as described herein above overlayed on a 3D image. It is noted however, that for the positional guidance as described herein before, the indicator need not be shown to a user per se. In such case the user may be provided the warning signal via visual, audible or haptic indication or combination of these.

FIG. 7 illustrates a valve view that triggers a fail-safe process to disable the laser device and provide sensory feedback to the operator, in accordance with an embodiment. The laser device 750 and grasping mechanism 705 is coupled with the ICE probe 710 with a field of view in the right atrium of heart 730. The failsafe can be triggered because of a deviation of the ICE probe 710 from the soft zone or a loss of view based on positional comparisons with the X-ray. For example, based on imaging features in X-ray and ultrasound, any deviation of the probe head or loss of view can be used to send signals to the anchoring system and either prompt user to reposition or automatically reposition the ICE probe.

In an exemplary embodiment, the sensory feedback device 700 includes a processor device to implement a machine learning (ML) algorithm or system to assist in a valve resection procedure. The ML algorithm can apply a Neural Network (NN) model to detect and predict three-dimensional landmarks in a valve resection procedure for proper localization. The NN model may be configured as a semi-supervised trained NN based on a set of ultrasound anatomical images generated in a prior valve resection procedure. In an exemplary embodiment, the sensory (processor) feedback device 700 can implement the NN model by processing a set of images of cross-sectional views of leaflets in the valve resection procedure to monitor a grasping operation of a leaflet by a grasping mechanism 705 and to provide confirmation of proper leaflet insertion during the grasping operation based on image comparisons of images of leaflet insertions during the grasping operation with cross-sectional views of leaflets contained in the NN model. The ML system with the processor device is configured to implement the NN model to track the motion of leaflets during the grasping operation for comparisons of aspects of leaflet motion to determine the proper leaflet insertion. The ML system with the processor device is configured to implement the NN model to compare pre-grasping leaflet motion versus post-grasp motion to determine the proper leaflet insertion.

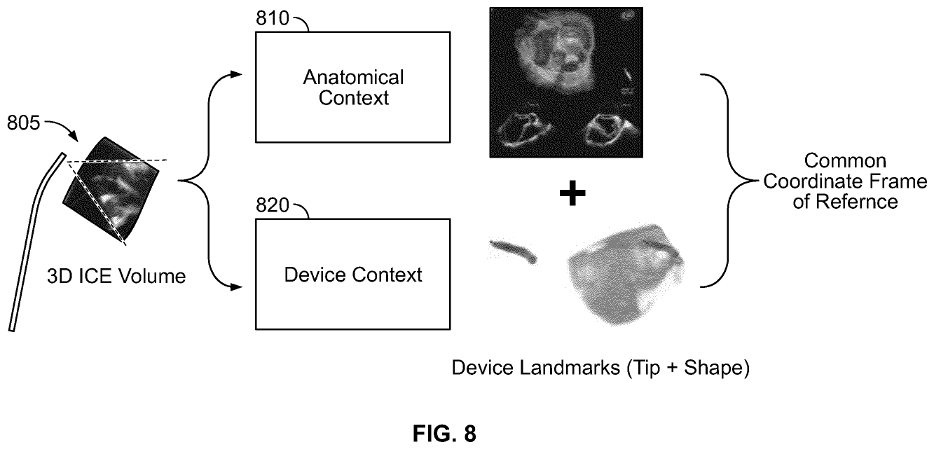

FIG. 8 illustrates a diagram of a common coordinate frame with a reference for navigating an ICE volume with anatomical landmarks in accordance with an embodiment. FIG. 8 illustrates a common coordinate frame of a reference to navigate in an ICE probe configured volume area with anatomical landmarks. In FIG. 8, processing module 810 provides an anatomical context with the anatomical landmarks and the common coordinate frame of reference. The processing module 820 provides the ICE probe context in the 3D ICE probe volume 805 for navigation of the laser catheter.

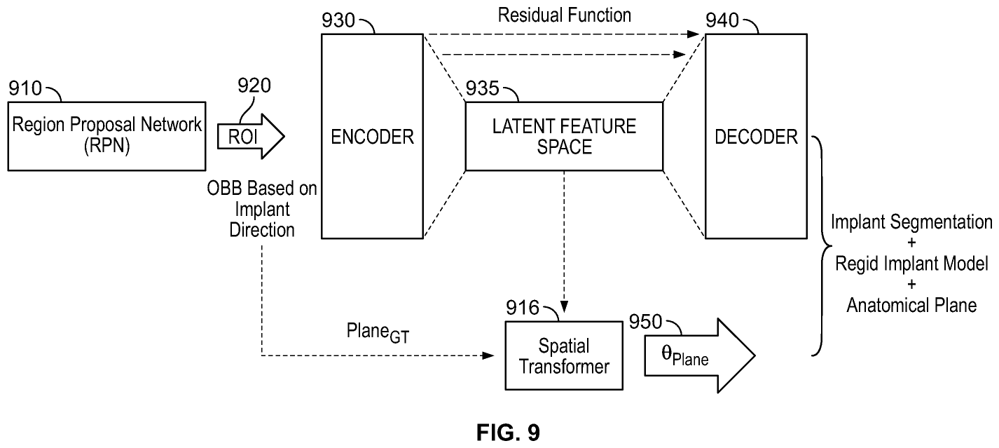

FIG. 9 illustrates a diagram of a combination of a neural network (NN) trained system in multi-task learning for predicting anatomical landmarks in the resection procedure in accordance with an embodiment. FIG. 9 includes a region proposal network (RPN) 910, that configures a region for interest 920 for an oriented bounding box (OBB) based on the determined implant direction and is sent to an encoder 930 to determine the latent feature space 935. This feature space is received by the decoder 940 that performs a series of functions that can include implant segmentation, rigid implant modelling, and anatomical plane configuration. Also, planar transforms are configured by the planar transformer 950 for displaying the two-dimensional (2D), 3D, and four-dimensional (4D) ultrasound images. The algorithm controller is designed to detect, localize, and classify valve and surrounding anatomy in a 3D ultrasound image to ensure the proper leaflet resection. The system can receive a set of 3D and time ultrasound images, receiving ground truth, manual expert annotations for key-points of valve anatomy and about labels associated with anatomy, and automated key-points and labels using a local fitting deformable geometric model. In an exemplary embodiment, the training of a machine learning model or neural network model using 3D ultrasound images to detect and predict 3D locations of anatomical landmarks, regress anatomical valve plane, generate candidate segmentation for fitting anatomical valve model, etc is implemented. This implementation may be in the form of a cascade of connected neural network models which are designed to execute the training of each neural network model in a semi-supervised multi-task learning paradigm.

In an exemplary embodiment, to specifically localize the valve, a region proposal network can be trained to detect and localize the subset of the image containing the valve. The output of the network is an oriented bounding box, the extent of which can be defined by the geometric limits of the valve or implant model. This localized sub-volume (SV) is then processed by an encoder-decoder network configuration for multi-task learning of latent space vectors which can be used to reconstruct a probabilistic segmentation and to train a spatial transformer for regressing a plane containing the anatomical reference (e.g., valve plane showing the cross-section of leaflets). The spatial transformer is also trained to generate a transformation matrix that can define a 2D plane used to sample the 3D sub-volume to produce an anatomical plane. This is training is performed by optimizing a loss function that minimizes the structural similarity index loss and means squared loss between the generated plane and ground truth plane sampled from labeled data. The ground truth images generated are based on standard 2D US images and the ground truth images can be added to the training process to improve the trained model. The network when applied can generate an anatomical plane of relevance and a candidate sparse segmentation. This information can reduce the burden of a large amount of labeled data. A combination of the anatomical plane and sparse segmentation mask can be used to initialize a deformable or rigid image-based model-fitting algorithm local anatomical coordinate frame of reference.

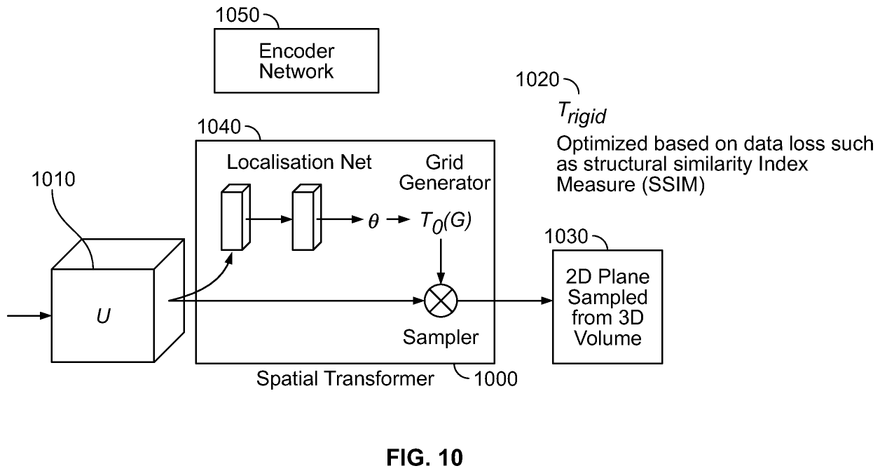

In FIG. 10 an exemplary diagram of a Spatial transformer network (STN) 1000 is illustrated that receives and consumes inputs from the encoder that defines a localization network. The latent feature vector generated by this process is condensed to regress a rigid transformation matrix Trigid 1020. The transformation matrix is used to transform a 2D identity plane within the span of the input 3D volume 1010. The plane generated is then applied to sample a 2D slice from the 3D volume. The STN 1000 is trained based on the global multi-task loss which includes image data loss based on metrics such as structural similarity index. The trained STN is capable to generate a 4×3 transformation matrix Trigid 1020 which defines a transformation 1040 to be applied to a 2D plane used to sample the 3D sub-volume to produce an anatomical plane 1030. The initial transformation 1040 is set as an identity matrix located at the center of the 3D sub-volume. The identity matrix is updated by optimizing a loss function that minimizes the structural similarity index loss and means squared loss between the generated plane and ground truth plane sampled from labeled data. Iteratively the (encoder) network 1050 trains to regress the pose of a 2D plane to output a pose that is closest and resembles the anatomic plane of interest from the 3D volume. Additionally, ground truth images received from standard 2D US images can be added to the training process. The network 1050 learns to generate an anatomical plane of relevance and a candidate sparse segmentation. This reduces the burden of a large amount of labeled data. A combination of the anatomical plane and sparse segmentation mask can be used to initialize a deformable or rigid image-based model-fitting algorithm local anatomical coordinate frame of reference.

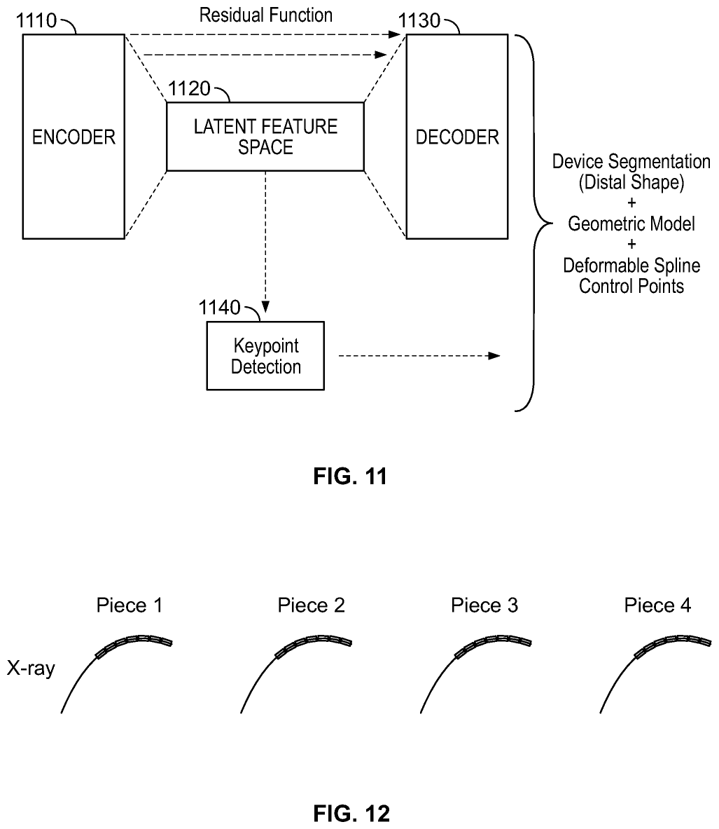

FIG. 11 illustrates an exemplary diagram of a machine learning system for enabling a type of multitask learning network for detecting devices in 3D ultrasound volumes, in accordance with an exemplary embodiment. The system includes an encoder 1110, a latent feature space 1120, a decoder 1130, and a key point detection module 1140 that enable device segmentation of the distal shape with a geometric model and the use of deformable spline control points.

The training of a machine learning model or neural network model using 3D ultrasound images can detect and predict 3D landmarks for non-anatomical features such as implants using co-registered 3D ultrasound and 2D x-ray data. The catheter-like devices often appear ambiguous in ultrasound images and can be misinterpreted for anatomical features when the devices are positioned closer to tissue. In a dynamic environment that is present during the tissue resection, discerning the ultrasound images can become even more challenging. An example of setting device tip location and distal end shape is to train a network for the multi-task learning paradigm. This training is performed either by fully supervised in a semi-supervised fashion where data labelling constraints are more relaxed. The typical architectures used for this are an Encoder-Decoder combination where convolutional filter banks decompose the input image into a feature vector that captures the minimum information required to extract the desired feature. In this multi-task learning paradigm, the latent features are then used to train a key point detection network, such as a 3D Yolo or convolution pose machines, which can provide potential candidate locations of the central axis of the catheter in ultrasound. The decoder part of the network is used to also reconstruct the learned features back up to the scale of the original image in the form of a probability map. This can be used to reconstruct a potential segmentation map of the image.

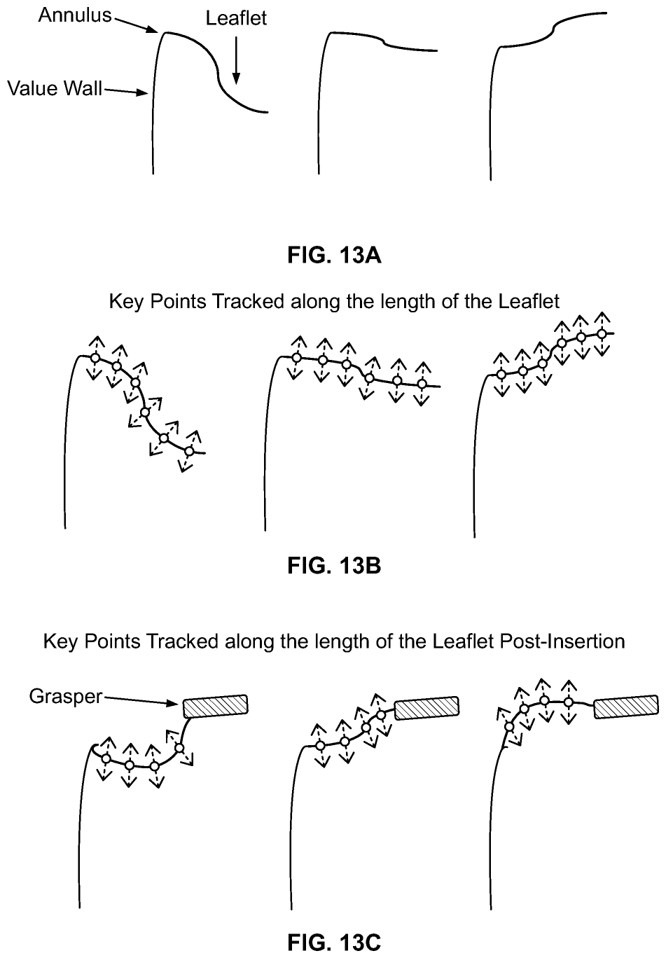

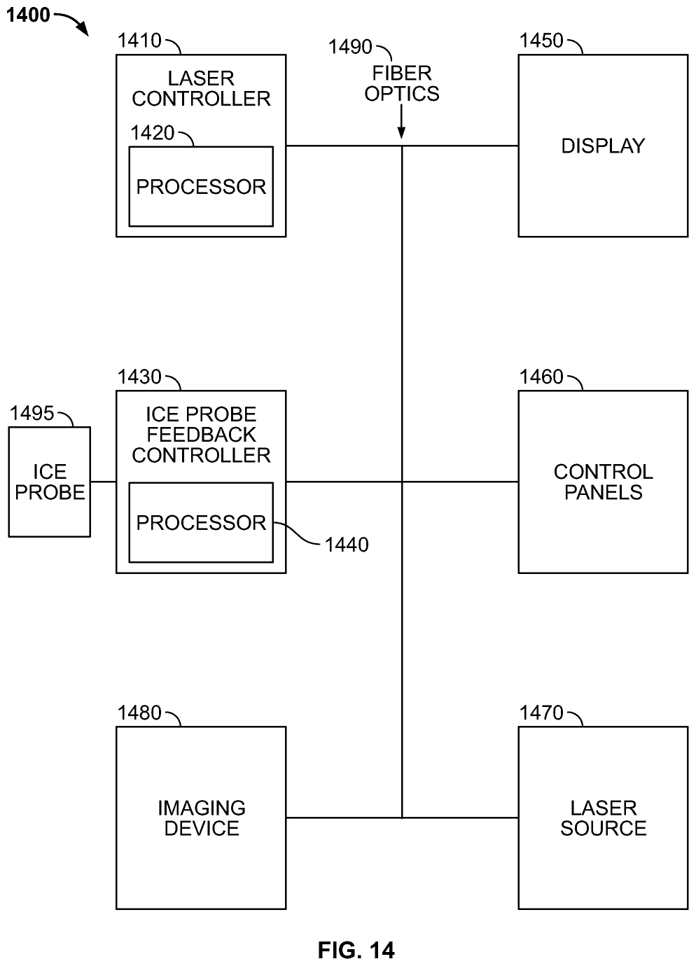

FIG. 12 illustrates an exemplary diagram of a piecewise approximation for combining landmarks from X-ray to ultrasound to improve the distal shape and catheter segmentation of catheter-like devices in accordance with an embodiment.

A critical part of the resection procedure is proper localization and grasping of the targeted leaflet. The localization of the leaflet is done as per the methods described in the previous section. Once this is done, the leaflet is imaged in an orthogonal cross-sectional view where grasping is monitored for proper leaflet insertion in the grasping mechanism. As a confirmation of proper leaflet insertion, the motion of the leaflet is tracked in the cross-sectional 2D imaging plane utilizing methods such as speckle tracking. Comparing pre-grasping leaflet motion via piecemeal portions in a series of parametric time-based models, particularly with respect to the tip of the leaflet, versus post-grasp motion can aid in determining the quality of insertion providing the clinician with the necessary confidence in advancing the laser re-sectioning device. In FIG. 12 the series of piecemeal approximations (Piece 1-4) of the movement of the ICE probe or other catheter-like devices can be fitted together to monitor the localization of the grasping operations in the leaflet ablation processes for the endoscopic procedure.

In an exemplary embodiment, the segmentation maps, as well as key point detection, tend to be noisy. But having this spatial information, it is possible to reduce the search space for local model-to-image-based registration techniques. Additionally, in X-ray imaging, the above-described network architecture can be utilized to perform keypoint detection, shape segmentation, and 3D pose regression based on training networks using synthetic images generated from DRR. By utilizing the known spatial relationship between ICE volume and corresponding X-ray image, the detection of device key points is projected along epipolar lines back to ultrasound space, the search space for geometric model localization is reduced. The information from X-ray space is then combined with ultrasound in a piecewise fashion which allows the model fitting to be a 3D parametric spline fitting problem. Additional contextual constraints can be added to the geometry of the device. For example, catheter-like devices often have radio-opaque markers that are spaced at a certain distance from the tip. This can add to improving the accuracy of the tip localization in ultrasound space.

FIGS. 2A, 13B, and 13C illustration exemplary diagrams of leaflet motion estimations for a leaflet resection procedure in accordance with an embodiment. In FIG. 13A there is illustrated a leaflet state over time; In FIG. 13B there is illustrated the motion of key points along the length of the leaflet and in FIG. 13C there is illustrated the restricted motion of leaflets after grasping by a tool. The processing unit (in FIG. 7) is capable of performing a series of steps in FIGS. 13A-C that includes accepting ultrasound images (2D, 3D, 2D+time, 3D+time) and executing conventional data (image, signals) processing algorithms and/or machine learning and deep learning algorithms.

In an exemplary embodiment, an algorithmic block applied by the processing unit is configured to detect leaflet key points, track leaflet key points in time, calculate leaflet length, analyze leaflet displacement, etc. The algorithm is designed to localize key features along the length of the leaflet in a 2D/3D cross-sectional view of the valve leaflet. Example of techniques which may be used, but not limited to, are SURF, SIFT, ORB, BRIK, MSER, HARRIS, etc. The key points can be localized in either single or multiple parallels or oblique imaging planes. For robustness, the detected key points can be clustered and resampled. The key points are localized in multiple frames along the time axis (e.g., previous, current, next). Next, the displacement of these key points is calculated between frames. The displacement may be calculated by treating this as a non-rigid image-to-image registration problem between subsequent frames, propagating through time. Owing to the repetitive cardiac motion, the average displacement of the leaflet can be calculated within a few cycles. Methods such as demon registration and its variation, sparse demons can be used for this purpose.

In an exemplary embodiment, a critical part of the resection procedure is proper localization and grasping of the targeted leaflet. The localization of the leaflet is done as per the methods described in the previous section. Once completed, the leaflet is imaged in an orthogonal cross-sectional view where grasping is monitored for proper leaflet insertion in the grasping mechanism. As a confirmation of proper leaflet insertion sent to a feedback device in FIG. 7), the motion of the leaflet is tracked in the cross-sectional 2D imaging plane utilizing methods such as speckle tracking. Comparing pre-grasping leaflet motion, particularly the tip of the leaflet, versus post-grasp motion can help determine the quality of insertion providing the clinician with the necessary confidence in advancing the laser re-sectioning device.

In an exemplary embodiment, in an alternate data-driven approach, a machine learning or neural network model can be trained to detect key points along with the leaflet in a frame-by-frame manner and then characterize the motion of key points between the frames. Examples of such a network are Yolo or convolutional pose machines. Typically, such methods require supervised training to perform well. In medical imaging, obtaining annotated data is challenging and is typically scarce depending on the modality in question. To overcome this, leaflet motion can be characterized by calculating the flow vector by employing neural models that can be trained in an unsupervised fashion.

The data used for training this network can be loops of leaflet motion within a cardiac cycle without grasping as well as with grasping. Here the neural network learns to generate flow vectors that map two temporally separated images to each other. The network backbone includes an encoder-decoder architecture that decomposes the input image to latent feature space and then reconstructs flow vectors. Two branches of the network are used with weight sharing between two temporally separated images. The network is trained using a combination of loss functions which enforce smoothness of the flow fields, and structural similarity between the pair of images after the new image is warped to the previous image using the flow field vectors. At inference time, the magnitude of the flow field vectors can be used to characterize before and after grasping leaflet motion.

In the implementation, a hybrid approach combining classical (a) and data-driven (b) can be used. Additionally, the cross-sectional 2D and 3D images can be sampled at multiple locations to improve the robustness of characterization. Additionally, flow and hemodynamic information can also be combined with image features to characterize the leaflet grasping and provide a confidence score. Additionally, during data acquisition for the training of the neural network, associated events-(1) grasping successfully and re-grasping for unsuccessful grasps and (2) depth of grasp/leaflet insertion can be tagged. This can be an additional input to classify flow vectors as grasped vs not grasped as well as providing metrics for the level of insertion and confidence.

The application phase controller block is configured to Receive 2D/3D+time ultrasound images, to calculate/Predict flow vectors and magnitude of leaflet motion, to predict the level of insertion of leaflet after grasping as confidence map or a numerical range. The successful evaluation of resection can be visualized by providing the ability to toggle a rendering overlay of the geometric model of the implanted valve as localized in the previous section over a live ICE image.

FIG. 14 illustrates a laser ablation, ICE probe feedback system, and imaging system 1400 according to some embodiments. Laser ablation controller 1410, ICE probe feedback system 1430, an imaging system 1480 which includes a laser generator 1470 coupled to a laser ablation controller 1410. The Controller 1410 includes one or more computing devices having a computer-readable medium programmed to control laser generator 1470, as described in the previous FIGS. 1-13. The controller 1410 may be internal or external to laser generator 1470. Laser generator 1470 may include an excimer laser or another suitable laser. In some embodiments, laser generator 130 produces light in the ultraviolet frequency range. The laser generator 1470 is connected with the proximal end of a laser catheter via a coupler. The distal end of the catheter may be inserted into a vessel or tissue of a human body. In some embodiments, system 1400 employs an ICE probe to guide laser light from the laser generator through a catheter toward a target area in the human body (i.e. target cardiac anatomy). Fiberoptic bundle 1490 includes any suitable number of optical fibers and, in some embodiments, to provide coupling between the various controller and devices in use.

The ICE probe feedback controller 1430 includes a processor 1440 with processing capability sufficient to implement various machine learning algorithms and process input data from an imaging device 1480. In addition, controller 1430 provides aural, audible, or other notifications of guidance as an ICE probe 1495 travels down a vessel lumen to a position that provides an optimal field of view of the cardiac autonomy (i.e., leaflets) during the tissue ablation.

Those of skill in the art will appreciate that the various illustrative logical blocks, modules, circuits, and algorithm steps described in connection with the embodiments disclosed herein may be implemented as electronic hardware, computer software, or combinations of both Some of the embodiments and implementations are described above in terms of functional and/or logical block components (or modules) and various processing steps.

However, it should be appreciated that such block components (or modules) may be realized by any number of hardware, software, and/or firmware components configured to perform the specified functions. To clearly illustrate the interchangeability of hardware, various illustrative components, blocks, modules, and steps have been described above generally in terms of their functionality. Whether such functionality is implemented as hardware or software depends upon the application and design constraints imposed on the overall system.

Skilled artisans may implement the described functionality in varying ways for each application, but such implementation decisions should not be interpreted as causing a departure from the scope of the present invention. In addition, those skilled in the art will appreciate that the embodiments described herein are merely exemplary implementations.

In this document, relational terms such as first and second, and the like may be used solely to distinguish one entity or action from another entity or action without necessarily requiring or implying any actual such relationship or order between such entities or actions. Numerical ordinals such as “first,” “second,” “third,” etc. simply denote different singles of a plurality and do not imply any order or sequence unless specifically defined by the claim language. The sequence of the text in any of the claims does not imply that process steps must be performed in a temporal or logical order according to such sequence unless it is specifically defined by the language of the claim. When “or” is used herein, it is the logical or mathematical or, also called the “inclusive or.” Accordingly, A or B is true for the three cases: A is true, B is true, and A and B are true. In some cases, the exclusive “or” is constructed with “and;” for example, “one from the set A and B” is true for the two cases: A is true, and B is true.

Furthermore, depending on the context, words such as “connect” or “coupled to” used in describing a relationship between different elements do not imply that a direct physical connection must be made between these elements. For example, two elements may be connected to each other physically, electronically, logically, or in any other manner, through one or more additional elements.

While at least one exemplary embodiment has been presented in the foregoing detailed description of the invention, it should be appreciated that a vast number of variations exist. It should also be appreciated that the exemplary embodiment or exemplary embodiments are only examples and are not intended to limit the scope, applicability, or configuration of the invention in any way. Rather, the foregoing detailed description will provide those skilled in the art with a convenient road map for implementing an exemplary embodiment of the invention. It is understood that various changes may be made in the function and arrangement of elements described in an exemplary embodiment without departing from the scope of the invention as set forth in the appended claims.

Further examples (to base clauses on) are provided herein below.

In one exemplary embodiment, an apparatus for use in an intracardiac procedure, such as for example a Transcatheter Aortic Valve Replacement (TAVR) procedure, is provided. The apparatus can include an intrabody ultrasound probe, such as for example an Intra-Cardiac Echography (“ICE”) probe and includes a processor device. The apparatus may include the intrabody ultrasound probe to be coupled to the processor device. The processor device configured to provide positional feedback, for example to a user or other system, about the intrabody ultrasound probe head position as the intrabody ultrasound probe head is positioned, for example manually or using a robotic system, within a cardiac anatomy. The processor device is configured to implement a model to provide guidance to position the intrabody ultrasound probe head based on anatomical recognition of a cardiac anatomy in which the intrabody ultrasound probe head is located at a position in the cardiac anatomy to enable capture a view of a target cardiac anatomy. Optionally also a catheter used while performing the intracardiac procedure is located in the cardiac anatomy and possibly recognized.

In at least one exemplary embodiment, an apparatus that is provided includes the processor device that is further configured to determine an initial position and a three-dimensional (3D) orientation of the intrabody ultrasound probe head within the cardiac anatomy to display a field of view (FOV) of the cardiac anatomy while performing the intracardiac procedure.

In at least one exemplary embodiment, an apparatus that is provided includes the processor device that is further configured to determine the position of the intrabody ultrasound probe head with a three-dimensional (3D) volume at a location-oriented in front of the intrabody ultrasound probe head; and check the 3D volume of the location-oriented in the front of the intrabody ultrasound probe head based on a set of multiple criteria that assess factors associated with the 3D volume, the cardiac anatomy, and the position of the catheter used in the intracardiac procedure.

In at least one exemplary embodiment, an apparatus that is provided includes the processor device that is further configured to: determine a 3D sub-volume within the 3D volume wherein the 3D sub-volume comprises a safe-zone and detect a deviation of the intrabody ultrasound probe head from the safe-zone configured within the 3D volume wherein the safe-zone is oriented in front of the ICE probe.

In at least one exemplary embodiment, an apparatus that is provided includes the processor device that is further configured to: provide notification via sensory feedback of the deviation of the intrabody ultrasound probe head from the safe zone.

In at least one exemplary embodiment, an apparatus that is provided includes the processor device that is further configured to: apply a registration algorithm to determine the 3D orientation of the intrabody ultrasound probe head within the cardiac anatomy.

In at least one exemplary embodiment, an apparatus that is provided includes the processor device that is further configured to record a spatial position and the 3D orientation of the intrabody ultrasound probe head as a combined reference position; plot a virtual 3D zone by comparing the combined reference position to a location in an X-ray image received by the feedback processor device to determine the safe-zone of the intrabody ultrasound probe head; and compare the movement of the intrabody ultrasound probe head based on the virtual 3D zone for any deviations of the intrabody ultrasound probe head to different locations within the cardiac anatomy.

In another exemplary embodiment, an apparatus to stabilize an intrabody ultrasound probe head, such as for example an Intracardiac Echocardiography (ICE) probe head, in an intracardiac procedure is provided. The apparatus includes a balloon device disposed about an elongated shaft section at the distal end of the intrabody ultrasound probe head at an outer surface for insertion with the distal end of the intrabody ultrasound probe head during the intracardiac procedure; an inflatable balloon disposed about a portion of the outer surface at the distal end of the elongated shaft section that is configured in a first state for insertion into the vessel lumen to travel down the vessel lumen, and a second state to stabilize the head of the intrabody ultrasound probe head; and in response to the inflatable balloon at the distal end of the elongated shaft section placed at a location about the head of the intrabody ultrasound probe head in the vessel lumen, the inflatable balloon is configured to stabilize the head of the intrabody ultrasound probe head in the second state by exerting a compression force to the distal end of the intrabody ultrasound probe head that includes the intrabody ultrasound probe head wherein the compression force is caused by inflation of the inflatable balloon sized to the vessel lumen and pressing against the intrabody ultrasound probe head.

In at least one exemplary embodiment, an apparatus that is provided includes the intrabody ultrasound probe head stabilized at a position in the vessel lumen to allow for a view of a target cardiac anatomy during the endoscopic procedure.

In at least one exemplary embodiment, an apparatus that is provided includes the inflatable balloon sized to about a diameter of the vessel lumen to stabilize the head of the intrabody ultrasound probe head in the second state.

In at least one exemplary embodiment, an apparatus that is provided includes the inflatable balloon of the balloon device with a compliant material that prevents overstretching of the vessel lumen in the second state.

In at least one exemplary embodiment, an apparatus that is provided includes the balloon device to stabilize the intrabody ultrasound probe head configured using an occlusion balloon type of device.

In at least one exemplary embodiment, an apparatus that is provided includes in response to the balloon device stabilizing the intrabody ultrasound probe head in the vessel lumen, a laser catheter is enabled to perform the ablation for the endoscopic surgery by a single user as further assistance of another user to position the intrabody ultrasound probe head to capture the view of the target anatomy during the endoscopic procedure is no longer require.

In at least one exemplary embodiment, an apparatus that is provided includes the balloon device with a compliant material that prevents overstretching of the vessel lumen when the inflatable balloon device is in the second state.

In at least one exemplary embodiment, an apparatus that is provided includes the balloon device configured to prevent movement of the intrabody ultrasound probe head in the vessel lumen by the compression force of the distal end of the ICE probe against the vessel lumen.

In at least one exemplary embodiment, an apparatus that is provided includes the balloon device is configured to prevent movement of the head of the intrabody ultrasound probe head caused by a set of actions that comprise blood flow, user interaction, and cardiac motion in the vessel lumen.

In summary of some examples the application provides systems and apparatuses for a resection procedure are provided. The apparatus includes an Intracardiac Echocardiography (ICE) probe for use in a Transcatheter Aortic Valve Replacement (TAVR) procedure, the ICE probe; and a processor device coupled to the ICE probe to provide positional feedback to a user about the ICE probe position as the ICE probe is positioned manually within cardiac anatomy wherein the processor device is configured to implement a model to provide guidance to manually position the ICE probe based on anatomical recognition of the cardiac anatomy wherein a manually positioned ICE probe is located at a position in the cardiac anatomy to enable capture of a view of a target cardiac anatomy in combination with the use of a catheter used while performing the TAVR procedure

1. An apparatus for use in an intracardiac procedure, the apparatus comprising:
a processor configured to:
provide positional feedback about a position of an intrabody ultrasound probe head as the intrabody ultrasound probe head is positioned within a cardiac anatomy to capture a view of a target anatomy of the cardiac anatomy;
generate the positional feedback based on a reference position of the ultrasound probe head within the anatomy wherein the reference position comprises a position from which the intracardiac ultrasound probe captures a desired view of the target anatomy using an ultrasound field of view; and
provide a feedback signal based on the positional feedback.

2. The apparatus of claim 1, wherein the reference position comprises a position with which the intracardiac ultrasound probe captures the desired view of the target anatomy and an intrabody tool for use during the intracardiac procedure.

3. The apparatus of claim 1, wherein the processor is further configured to generate the positional feedback further based on structural characteristics of the target anatomy and one or more of: the field of view and the desired view.

4. The apparatus of claim 1, wherein the processor is further configured to:
predicting a safe zone in the form of a virtual volume around the reference position, the safe zone encompassing positions the ultrasound probe head can reside in during the cardiac procedure while providing an acceptable one of the desired view; and
determine whether a deviation of the current position from the safe zone occurs.

5. The apparatus of claim 1, wherein the processor is configured to determine a similarity measure representative of a similarity between a current view of the intrabody target obtained with the intrabody ultrasound probe and a predetermined view of the target anatomy and determine the current view to be the desired view based on the similarity measure to therewith determine the reference position to be the current position pertaining to the corresponding current view.

6. The apparatus of claim 5, wherein the processor is configured to determine the safe zone based on the similarity measure as the volume of the safe zone increases with increasing similarity measure.

7. The apparatus of claim 4, wherein volume of the soft zone is determined further based on a set of positional tolerances defined to accommodate an extent of motion of the intrabody ultrasound probe head without loss of the desired viewed of the target anatomy.

8. The apparatus of claim 4, wherein the processor is further configured to:
receive an image of an imaging device;
generate a representation of the safe zone for overlay with the image; and
generate the positional feedback based on the image and the representation of the safe zone.

9. The apparatus of claim 1, wherein the processor is further configured to:
generate, during the intracardiac procedure, the positional feedback comprising one or more of: a representation of the safe zone; the deviation of the current position from the safe zone, instructions for repositioning the intrabody ICE probe head to within the safe zone and instructions to reacquire a reference position.

10. A computer implemented method for use during an intracardiac procedure comprising:
providing positional feedback about a position of an intrabody ultrasound probe head as the intrabody ultrasound probe head is positioned within a cardiac anatomy to capture a target anatomy of the cardiac anatomy;
using a model, generating the positional feedback based on a reference position of the ultrasound probe head within the anatomy, wherein the reference position comprises a position from which the intracardiac ultrasound probe captures an desired view comprising the target anatomy to be treated during the intracardiac procedure using an ultrasound field of view; and
provide a feedback signal based on the positional feedback

11. A non-transitory computer-readable storage medium having stored a computer program comprising instructions which, when executed by a processor, cause the processor to:
provide positional feedback about a position of an intrabody ultrasound probe head as the intrabody ultrasound probe head is positioned within a cardiac anatomy to capture a view of a target anatomy of the cardiac anatomy;
generate the positional feedback based on a reference position of the ultrasound probe head within the anatomy wherein the reference position comprises a position from which the intracardiac ultrasound probe captures a desired view of the target anatomy using an ultrasound field of view; and
provide a feedback signal based on the positional feedback.

12. The apparatus of claim 1, further comprising a system for stabilizing a probe head at the distal end of an Intracardiac Echocardiography (ICE) probe in a vessel lumen during an intracardiac procedure, the system comprising an inflatable balloon device disposable about the probe head upon deployment within the vessel lumen; the inflatable balloon having a first state for insertion of the inflatable balloon into the vessel lumen together with the probe head during the intracardiac procedure and the inflatable balloon device having a second state to stabilize the probe head within the lumen, wherein the system is configured such that the inflatable balloon can be inflated to cause it to change from the first state to the second state while deployed in the vessel lumen together with the probe head to therewith cause the inflatable balloon to exert a compression force to part of the probe head and at least part of the vessel lumen wall surrounding the probe head to thereby provide the stabilization.

13. The apparatus of claim 12, wherein the inflatable balloon comprises a first opening at its distal end and a second opening at its proximal end and a lumen connecting the first and the second opening, the lumen being configured to accommodate at least a part of the probe head.

14. The apparatus of claim 12, wherein the inflatable balloon of the balloon device comprises a compliant material that prevents overstretching of the vessel lumen when the inflatable balloon is in the second state.

15. The apparatus of claim 12, wherein the inflatable balloon device is an occlusion balloon type of device.

16. The apparatus of claim 12, wherein the inflatable balloon is designed to be shaped in the second state such that when deployed in the vessel lumen in the second state a channel for blood flow extends from a proximal end to a distal end of the balloon.

17. The apparatus of claim 12, wherein stabilizing the probe head comprises a reduction or prevention of motion of the probe head caused by one or more actions chosen from the group consisting of: blood flow in the vessel lumen, cardiac motion, and user interaction.

18. The apparatus of claim 12, wherein in response to the balloon device stabilizing the probe head in the vessel lumen, a laser catheter is enabled to perform ablation for the intracardiac procedure by a single user.

19. A The apparatus of claim 1, further comprising a system to assist in a valve resection procedure comprising:
a Neural Network (NN) model to detect and to predict a plurality of three-dimensional landmarks in a valve resection procedure for proper localization wherein the NN model is a semi-supervised trained NN based on a set of ultrasound anatomical images generated in a prior valve resection procedure; and
the processor further configured to implement the NN model by processing a set of images of cross-sectional views of leaflets in the valve resection procedure to monitor a grasping operation of a leaflet by a grasping mechanism and to provide confirmation of proper leaflet insertion during the grasping operation based on image comparisons of images of leaflet insertions during the grasping operation with cross-sectional views of leaflets contained in the NN model.

20. The apparatus of claim 19, wherein the processor is configured to implement the NN model to track movement of leaflets during the grasping operation for comparisons of aspects of leaflet motion to determine the proper leaflet insertion.

21. The apparatus of claim 20, wherein the processor is configured to implement the NN model to compare pre-grasping leaflet motion versus post-grasp motion to determine the proper leaflet insertion.

22. The system of claim 21, wherein the processor is configured to implement the NN model to estimate pre-grasping motion versus post-grasping motion to determine in advance of the proper leaflet insertion.
